(19) United States
(12) Patent Application Publication
(10) Pub. No.: US 2010/0137345 A1
(43) Pub. Date: Jun. 3, 2010

(54) PROPHYLACTIC AND THERAPEUTIC USE OF SIRTUIN INHIBITORS IN TNF-ALPHA MEDIATED PATHOLOGIES

(75) Inventors: Oberdan Leo, Wezembeek-Oppem (BE); Mara Galli, Bruxelles (BE); Frédéric Van Gool, Kraainem (BE)

Correspondence Address: KNOBBE MARTENS OLSON & BEAR LLP, 2040 MAIN STREET, FOURTEENTH FLOOR, IRVINE, CA, 92614, US

(73) Assignee: Universite Libre de Bruxelles, Bruxelles (BE)

(21) Appl. No.: 12/599,910

(22) PCT Filed: May 14, 2008
(86) PCT No.: PCT/EP2008/055880

§ 371 Date: Nov. 12, 2009

(30) Foreign Application Priority Data
May 14, 2007 (EP) 07108126.9

(51) Int. Cl. A61K 31/513; C07C 237/30; C07D 239/56; C07D 311/20; A61K 31/166; A61K 31/37; A61P 31/00; A61P 37/00; A61P 37/08; A61P 27/02; A61P 17/00; A61P 11/02; A61P 25/00; A61P 11/00; A61P 11/06; A61P 29/00; A61P 1/00; C12N 5/071

(52) U.S. Cl. 514/274; 564/167; 544/314; 549/290; 514/619; 514/457; 435/375

(57) ABSTRACT

The invention relates to the use of sirtuin inhibitors for reducing TNF-alpha production by cells and organisms. The invention also concerns prophylactic and therapeutic applications of sirtuin inhibitors in TNF-alpha mediated pathologies, such as various inflammatory and autoimmune disorders.

### FIELD OF THE INVENTION

The invention relates to the use of sirtuin inhibitors for reducing TNF-alpha production by cells and organisms. The invention also concerns prophylactic and therapeutic applications of sirtuin inhibitors in TNF-alpha mediated pathologies, such as various inflammatory and autoimmune disorders.

### BACKGROUND OF THE INVENTION

Tumor necrosis factor alpha (TNF-alpha, TNF-α), also known as cachectin, is a regulatory cytokine secreted inter alia by monocytes/macrophages, neutrophils, activated lymphocytes, NK cells, LAK cells, astrocytes, endothelial cells, smooth muscle cells, and some transformed cells. TNF-alpha regulates a wide variety of biological processes, including among others: cytotoxic effects against tumour cells, activation of neutrophils, inhibition or stimulation of proliferation of normal cells, modulation of immuno-inflammatory, immuno-regulatory, and anti-viral responses, as well as modulation of lipid metabolism, coagulation and insulin resistance. TNF-alpha is a central mediator of local and systemic inflammation.

Excessive or unchecked production of TNF-alpha may, however, be injurious to the host and may cause or contribute to various disease states, including inter alia cachexia associated with cancer or infectious diseases such as AIDS, endotoxin-induced shock, septic shock syndrome, systemic inflammatory response syndrome (SIRS), multiple organ dysfunction syndrome (MODS), chronic inflammatory and autoimmune diseases, such as, e.g., rheumatoid arthritis, inflammatory bowel disease (IBD) encompassing ulcerative colitis and Crohn's disease, osteoarthritis, Kawasaki's disease, multiple sclerosis (MS), Behchet's disease, systemic lupus erythematosus (SLE) and others, graft versus host pathologies, cerebral malaria, meningococcal meningitis, and type-II diabetes.

Given the central role of TNF-alpha in regulating normal inflammation processes as well as its involvement in the aetiology of numerous pathological conditions, modulators of TNF-alpha production and/or activity are highly desired as pharmaceutical agents. Previously, monoclonal anti-TNF-alpha antibodies adalimumab, etanercept and infliximab have been indicated for the treatment of one or more of rheumatoid arthritis, psoriatic arthritis, psoriasis, ankylosing spondylitis or Crohn's disease. Nevertheless, the need for further and improved TNF-alpha modulators is evident.

Sirtuins are a family of proteins related to the founding member of the family—the silent information regulator 2 protein (Sir2p) of Saccharomyces cerevisiae, a nicotinamide adenine dinucleotide (NAD⁺)-dependent histone deacetylase (HDAC) regulating chromatin silencing. Mammals contain at least seven sirtuin homologues numbered SIRT1 to SIRT7, characterised by significant sequence homology, particularly within their conserved NAD⁺ binding domains (Frye 2000. Biochem Biophys Res Commun 273: 793-798). Sirtuins usually possess NAD⁺-dependent deacetylase activity which removes acetyl groups from Ac-Lys of histones or non-histone protein substrates, while producing nicotinamide and acetyl ester metabolites 2′-O- and 3′-O-acetyl-ADP ribose (AADPR). AADPR may themselves function as second messengers in cells. Other sirtuins, e.g., SIRT4 and SIRT6, may lack significant deacetylase activity but display a robust NAD⁺-dependent ADP-ribosyl transferase activity (Liszt et al. 2005. J Biol Chem 280: 21313-21320).

Previous reports suggested that members of the sirtuin family could negatively regulate inflammatory responses. Nayagam et al. 2006 (J Biomol Screen 11: 959-67) proposed that activators of SIRT1 had anti-inflammatory properties through suppressing TNF-alpha. Yang et al. 2007 (Am J Physiol Lung Cell Mol Physiol 292: L567-76) indicated that sirtinol, an inhibitor of SIRT1, could increase the release of inflammatory cytokines induced by cigarette smoke extracts.

Several publications (WO 2007/007327, EP 0 486 809, WO 94/08574, U.S. Pat. No. 5,158,940, WO 02/071062, WO 98/03178 and EP 0 183 352) appear to disclose or suggest the administration of suramin in some conditions and diseases of interest to the present invention, or exposure of cells in vitro to suramin.

However, suramin (a highly charged polysulphonated derivative of urea which is mainly used for the treatment of African trypanosomiasis and onchocerciasis) is known to be a highly non-selective drug able to interfere with numerous extracellular protein-protein interactions. For example, suramin can bind to a variety of growth factors including platelet-derived growth factors (Hosang 1985. J Cell Biochem 29: 265-273), fibroblast growth factors (Rifkin & Moscatelli 1989. J Cell Biol 109: 1-6) and other cellular receptors. Further, suramin is also known to play a role in follicular lymphoma, angiogenesis, AIDS and several kinds of cancers such as prostate, renal cells and breast cancer. Suramin also displays ability to inhibit the myotoxic activity of different Crotalidae venoms.

In view of this wide spectrum of activities, suramin is often used as a non-specific inhibitor to evaluate whether a given biological response is mediated by protein-protein interactions. Moreover, due to its large and charged structure, suramin is a non-membrane permeable compound and its effects are therefore restricted to modulating extracellular protein-protein interactions.

Although suramin was also found to inhibit SIRT1 enzymatic activity (Howitz et al. 2003. Nature 425: 191-6), this evidently represents only one aspect among the plethora of its activities and potential interactions. In view thereof, the above documents cannot suggest to use sirtuin inhibitors other than suramin for inhibiting TNF-alpha production in isolated cells or in subjects, and for treating TNF-alpha mediated pathologies particularly associated with excessive or deregulated, systemic or local production of TNF-alpha in subjects. Moreover, due to the solely extracellular action of suramin, the above documents cannot suggest the use of cell-permeable sirtuin inhibitors for the herein intended uses.

Particularly noted is further that: WO 2007/007327 suggests that suramin acts as a potential inhibitor of PR-3 protease, thereby diminishing activation of pro-IL-32 and in turn synthesis of TNF-α; EP 0 486 809, WO 94/08574, U.S. Pat. No. 5,158,940 suggest that suramin blocks binding of TNF-α to its membrane receptor, thus inhibiting TNF-α biological activity; WO 02/071062 suggests that suramin antagonises nucleoside triphosphate disphosphohydrolase (NTPDase), thereby preventing inactivation of pro-inflammatory nucleotides; WO 98/03178 describes that suramin can inhibit PY2 receptors by binding to their extracellular portion; and EP 0 183 352 suggests that suramin may inhibit viral infectivity.

Given the known heterogeneity and non-specificity of suramin actions, also reflected in the variety of its effects described in the above documents, one would not reasonably assume that such actions would be due to its sirtuin inhibitor activity, and that other sirtuin inhibitors could achieve similar effects.

Further, WO 2007/014327 suggests to treat protein conformation disorders (PCD) using sirtinol; Heltweg et al. 2006 (Cancer Research 66: 4368-4377) suggests to use sirtuin inhibitors to reduce tumour cell growth in vitro and in vivo; and Yeung et al. 2004 (EMBO J 23: 2369-2380) demonstrate the ability of SIRT1 to modulate TNF-α-induced cell death. None of these studies suggests to use such sirtuin inhibitors for inhibiting TNF-alpha production in isolated cells or in subjects, and for treating TNF-alpha mediated pathologies associated with excessive or deregulated, systemic or local production of TNF-alpha in subjects as particularly contemplated in the present invention.

### SUMMARY OF THE INVENTION

The present invention addresses the existing need for further or improved modulators of TNF-alpha. Such modulators may be particularly useful for inter alia the prevention and treatment of TNF-alpha mediated pathologies. More specifically, the inventors have realised that sirtuin inhibitors can unexpectedly diminish the production of TNF-alpha by cells and organisms.

Accordingly, in an aspect the invention provides a method for reducing the production of TNF-alpha in cells in vitro, comprising exposing said cells to an inhibitor of one or more sirtuins.

A further aspect provides a method for reducing the production of TNF-alpha in cells in vitro, comprising exposing said cells to an inhibitor of one or more sirtuins other than suramin.

Another aspect provides a method for reducing the production of TNF-alpha in cells in vitro, comprising exposing said cells to a cell-permeable inhibitor of one or more sirtuins. Without being limited to any hypothesis or presumption, the inventors believe that intracellular action of sirtuin inhibitors may be required or favoured to achieve the desired effects on TNF-alpha production in cells in vitro, in vivo or in subjects.

In further aspects, the invention provides:—an inhibitor of one or more sirtuins for reducing the production of TNF-alpha in a subject;—the use of an inhibitor of one or more sirtuins for the manufacture of a medicament for reducing the production of TNF-alpha in a subject;—a method for reducing the production of TNF-alpha in a subject in need thereof, comprising administering to said subject an inhibitor of one or more sirtuins in an amount effective to achieve said reducing.

In still further aspects, the invention provides:—an inhibitor of one or more sirtuins other than suramin for reducing the production of TNF-alpha in a subject;—the use of an inhibitor of one or more sirtuins other than suramin for the manufacture of a medicament for reducing the production of TNF-alpha in a subject;—a method for reducing the production of TNF-alpha in a subject in need thereof, comprising administering to said subject an inhibitor of one or more sirtuins other than suramin in an amount effective to achieve said reducing.

In other aspects, the invention provides:—a cell-permeable inhibitor of one or more sirtuins for reducing the production of TNF-alpha in a subject;—the use of a cell-permeable inhibitor of one or more sirtuins for the manufacture of a medicament for reducing the production of TNF-alpha in a subject;—a method for reducing the production of TNF-alpha in a subject in need thereof, comprising administering to said subject a cell-permeable inhibitor of one or more sirtuins in an amount effective to achieve said reducing.

As noted, TNF-alpha may act as a stimulator of local and systemic inflammation. Hence, in further aspects, the invention provides:—an inhibitor of one or more sirtuins for use in reducing local or systemic inflammation in a subject;—the use of an inhibitor of one or more sirtuins for the manufacture of a medicament for reducing local or systemic inflammation in a subject;—a method for reducing local or systemic inflammation in a subject in need of said reducing, comprising administering to said subject a therapeutically effective amount of an inhibitor of one or more sirtuins.

Also in aspects, the invention provides:—an inhibitor of one or more sirtuins other than suramin for use in reducing local or systemic inflammation in a subject;—the use of an inhibitor of one or more sirtuins other than suramin for the manufacture of a medicament for reducing local or systemic inflammation in a subject;—a method for reducing local or systemic inflammation in a subject in need of said reducing, comprising administering to said subject a therapeutically effective amount of an inhibitor of one or more sirtuins other than suramin.

In further aspects, the invention provides:—a cell-permeable inhibitor of one or more sirtuins for use in reducing local or systemic inflammation in a subject;—the use of a cell-permeable inhibitor of one or more sirtuins for the manufacture of a medicament for reducing local or systemic inflammation in a subject;—a method for reducing local or systemic inflammation in a subject in need of said reducing, comprising administering to said subject a therapeutically effective amount of a cell-permeable inhibitor of one or more sirtuins.

In related aspects, the invention provides:—an inhibitor of one or more sirtuins for use in the treatment of a TNF-alpha mediated pathology;—the use of an inhibitor of one or more sirtuins for the manufacture of a medicament for the treatment of a TNF-alpha mediated pathology;—a method for treating a TNF-alpha mediated pathology in a subject in need of said treatment, comprising administering to said subject a therapeutically effective amount of an inhibitor of one or more sirtuins. “TNF-alpha mediated pathology” encompasses any pathological condition of a part, tissue, organ or system of a subject (i.e., a disease or disorder), wherein local or systemic excessive or deregulated production of TNF-alpha in the subject contributes to the aetiology of the disease or underlies one or more of its symptoms. Hence, the term includes any pathological condition the prevention or treatment of which can benefit from inhibition of TNF-alpha production or activity in so affected subject. Accordingly, the sirtuin inhibitors of the present invention are in particular for treating a TNF-alpha mediated pathology in a subject having local or systemic excessive or deregulated production of TNF-alpha.

In further related aspects, the invention provides:—an inhibitor of one or more sirtuins other than suramin for use in the treatment of a TNF-alpha mediated pathology;—the use of an inhibitor of one or more sirtuins other than suramin for the manufacture of a medicament for the treatment of a TNF-alpha mediated pathology;—a method for treating a TNF-alpha mediated pathology in a subject in need of said treatment, comprising administering to said subject a therapeutically effective amount of an inhibitor of one or more sirtuins other than suramin.

In aspects, the invention provides:—a cell-permeable inhibitor of one or more sirtuins for use in the treatment of a TNF-alpha mediated pathology;—the use of a cell-permeable inhibitor of one or more sirtuins for the manufacture of a medicament for the treatment of a TNF-alpha mediated pathology;—a method for treating a TNF-alpha mediated pathology in a subject in need of said treatment, comprising administering to said subject a therapeutically effective amount of a cell-permeable inhibitor of one or more sirtuins.

In further related aspects, the invention also provides:—a pharmaceutical composition comprising an inhibitor of one or more sirtuins and one or more pharmaceutically acceptable carriers for use in reducing local or systemic inflammation in a subject, or for use in the treatment of a TNF-alpha mediated pathology;—a method for reducing local or systemic inflammation in a subject in need of said reducing, or for treating a TNF-alpha mediated pathology in a subject in need of said treatment, comprising administering to said subject a pharmaceutical composition comprising a therapeutically effective amount of an inhibitor of one or more sirtuins and pharmaceutically acceptable one or more carriers. The invention also discloses said pharmaceutical compositions as such.

In related aspects, the invention also provides:—a pharmaceutical composition comprising an inhibitor of one or more sirtuins other than suramin and one or more pharmaceutically acceptable carriers for use in reducing local or systemic inflammation in a subject, or for use in the treatment of a TNF-alpha mediated pathology;—a method for reducing local or systemic inflammation in a subject in need of said reducing, or for treating a TNF-alpha mediated pathology in a subject in need of said treatment, comprising administering to said subject a pharmaceutical composition comprising a therapeutically effective amount of an inhibitor of one or more sirtuins other than suramin and pharmaceutically acceptable one or more carriers. The invention also discloses said pharmaceutical compositions as such.

Also provided are:—a pharmaceutical composition comprising a cell-permeable inhibitor of one or more sirtuins and one or more pharmaceutically acceptable carriers for use in reducing local or systemic inflammation in a subject, or for use in the treatment of a TNF-alpha mediated pathology;—a method for reducing local or systemic inflammation in a subject in need of said reducing, or for treating a TNF-alpha mediated pathology in a subject in need of said treatment, comprising administering to said subject a pharmaceutical composition comprising a therapeutically effective amount of a cell-permeable inhibitor of one or more sirtuins and pharmaceutically acceptable one or more carriers. The invention also discloses said pharmaceutical compositions as such.

These and further aspects and preferred embodiments of the invention are described in the following sections and in the appended claims.

### BRIEF DESCRIPTION OF FIGURES

FIG. 1 illustrates downregulation of TNF-alpha secretion by sirtuin inhibitors (sirtinol, cambinol) in murine dendritic cells (A) and murine macrophage-like cell line RAW264.7 (B).

FIG. 2 illustrates that sirtuin inhibitors (sirtinol, cambinol) do not generally inhibit protein synthesis nor induce cell death in murine dendritic cells.

FIG. 3 illustrates that sirtuin inhibitors (sirtinol, A; cambinol, B) may downregulate TNF-alpha cytokine production in RAW264.7 cells without significantly affecting the intracellular accumulation of TNF-alpha mRNA.

FIG. 4 illustrates that overexpression of SIRT1 or SIRT6 increases TNF-alpha cytokine production in cells of human HeLa cell line transiently transfected with a TNF-alpha-encoding plasmid.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less from the specified value, insofar such variations are appropriate to perform in the disclosed invention. It is to be understood that the value to which the modifier “about” refers is itself also specifically, and preferably, disclosed.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. When specific terms are defined in connection with a particular aspect or embodiment, such denotation or connotation is meant to apply throughout this specification, i.e., also in the context of other aspects or embodiments, unless otherwise stated.

All documents cited in the present specification are incorporated by reference in their entirety.

The term “tumor necrosis factor alpha” abbreviated “TNF-alpha” or “TNF-α” refers to any proteins know under these denotations in the art. In particular, the term encompasses endogenous TNF-alpha of any organism where such is present, and preferably of animals or humans. By means of example and not limitation, human TNF-alpha may encompass endogenous proteins as disclosed in inter alia Pennica et al. 1984 (Nature 312: 724-9) and in the UniProtKB/Swiss-Prot database (http://www.expasy.org/uniprot/) entry P01375, as well as any sequence variants thereof due to normal sequence polymorphism between and within human populations. By means of further non-limiting examples, the term may encompass endogenous TNF-alpha proteins as annotated in the UniProtKB/Swiss-Prot database for bovine (Q06599), dog (P51742), goat (P13296), guinea pig (P51435), cat (P19101), horse (P29553), mouse (P06804), chimp (Q8HZD9), pig (P23563), rabbit (P04924), rat (P16599) and others, as well as any sequence variants thereof due to normal sequence polymorphism between and within populations of each respective species.

Further, the term TNF-alpha particularly encompasses the soluble, secreted cytokine form of TNF-alpha, including monomeric as well as, preferably, the typically more active trimeric forms thereof (see, e.g., Smith & Baglioni 1987. J Biol Chem 262: 6951-4). The primary amino acid sequences of soluble forms of endogenous TNF-alpha are indicated in the above mentioned UniProtKB/Swiss-Prot database entries for the respective exemplified organisms. In addition, the term TNF-alpha may also encompass membrane-bound forms of TNF expressed on the surface of some cell types (see, e.g., Kriegler et al. 1988. Cell 53: 45-53).

Further, the term TNF-alpha may also encompass synthetic or recombinant proteins whose primary amino acid sequence is identical or substantially identical (“substantially identical”, as used throughout this specification, generally refers to ≧80%, e.g., ≧85%, preferably ≧90%, more preferably ≧95%, even more preferably ≧98% or ≧99% identical) to the sequence of an endogenous TNF-alpha, as determined using, e.g., the “Blast 2 sequences” algorithm described by Tatusova & Madden 1999 (FEMS Microbiol Lett 174: 247-250), and which retain biological activity identical or substantially identical to the respective endogenous TNF-alpha, as determined using, e.g., the cytotoxicity tests described by Flick & Gifford 1984 (J Immunol Methods 68: 167-75).

Nevertheless, as will appear from the context of aspects and embodiments of the present invention, the term TNF-alpha may mostly refer herein to endogenous TNF-alpha, soluble and/or membrane bound, preferably soluble, produced by cells, tissues, organs or organisms, the production of which can be affected by methods and reagents of the invention.

The term “reducing the production of TNF-alpha” as used throughout this specification means effecting a statistically significant decrease in the amount of biologically active TNF-alpha produced by cells, tissues, organs or organisms per unit of time. Whereas the term encompasses any extent of such decrease, the latter may preferably be by at least about 10%, e.g., by at least about 20%, more preferably by at least about 30%, e.g., by at least about 40%, even more preferably by at least about 50%, e.g., by at least 60%, still more preferably by at least 70%, e.g., by at least about 80%, and very preferably by at least about 90%, e.g., by at least about 95% or by about 100%, relative to the amount of biologically active TNF-alpha produced by said cells, tissues, organs or organisms without (e.g., prior to) effecting said decrease. The production of TNF-alpha may be suitably determined and compared using quantification methods that measure either the steady-state concentration or the appearance in time (pulse-chase) of TNF-alpha. Such methods are routinely known in the art, and include, e.g., ELISA, RIA, immuno-precipitation, Western blotting, etc. Biological activity of TNF-alpha may be tested, e.g., as in Flick & Gifford 1984 (supra).

In an embodiment, said decrease in production of biologically active TNF-alpha may be achieved for the soluble, secreted cytokine form of TNF-alpha as well as for the membrane-bound form of TNF-alpha (Kriegler et al. 1988, supra). In preferred embodiments, said decrease in production may be achieved for at least (or only for) the soluble, secreted cytokine form of TNF-alpha, which is particularly involved in various cell signalling and regulatory processes.

A reduction in the amount of biologically active TNF-alpha produced by cells, a part, tissue or organ of an organism may lead to a statistically significant decrease in the concentration of biologically active TNF-alpha in (the surroundings of) said cells or within said part, tissue or organ (i.e., locally) and/or may cause a statistically significant reduction in the concentration of biologically active TNF-alpha in the whole organism (i.e., systemically), e.g., in blood, plasma and/or other bodily fluids, e.g., lymph or CSF, of said organism. While any extent of local or systemic decrease in TNF-alpha concentration is contemplated, it may preferably be by at least about 10%, e.g., by at least about 20%, more preferably by at least about 30%, e.g., by at least about 40%, even more preferably by at least about 50%, e.g., by at least 60%, still more preferably by at least 70%, e.g., by at least about 80%, and very preferably by at least about 90%, e.g., by at least about 95% or by about 100%, relative to the local or systemic concentration of biologically active TNF-alpha, respectively, without (e.g., prior to) effecting the reduction in the amount of produced biologically active TNF-alpha.

A reduction in the amount of biologically active TNF-alpha produced by cells in vitro may lead to a statistically significant decrease in the concentration of biologically active TNF-alpha in the cells or surroundings thereof, e.g., in the cell culture medium, preferably by the above listed decrements.

Whereas the agents of the present invention clearly achieve reduced production of biologically active TNF-alpha, the precise cellular mechanism(s) underlying said reduction is of lesser concern and may be any, such as, without limitation: diminished transcription, splicing, nuclear export or stability of TNF-alpha mRNA, diminished translation of TNF-alpha mRNA, diminished or impaired cell sorting, folding, proteolytic processing, secretion or trimerisation of soluble TNF-alpha protein, or diminished or impaired sorting, folding, membrane insertion or glycosylation of membrane-bound TNF-alpha, etc. Without limitation, data obtained by the inventors may indicate that the agents of the invention could impinge on one or more post-transcriptional processing steps leading to mature TNF-alpha.

The terms “sirtuin” or “SIRT” generally refer to any protein orthologous to the Sir2p protein of Saccharomyces cerevisiae (Frye 2000, supra). In particular, the terms encompass Sir2p orthologues of animals, preferably of mammals or humans, and more specifically include the sirtuins SIRT1, SIRT2, SIRT3, SIRT4, SIRT5, SIRT6 and SIRT7 of humans and any non-human animals, preferably mammals, where such are present. By means of example and not limitation, the term encompasses human SIRT proteins as annotated in the UniProtKB/Swiss-Prot database: SIRT1 (Q96EB6), SIRT2 (Q8IXJ6), SIRT3 (Q9NTG7), SIRT4 (Q9Y6E7), SIRT5 (Q9NXA8), SIRT6 (Q8N6T7) and SIRT7 (Q9NRC8), and any sequence variants thereof due to normal sequence polymorphism between and within human populations. By means of further non-limiting examples, the term encompasses SIRT from further animals, more preferably mammals, as annotated in the UniProtKB/Swiss-Prot database, such as: mouse (SIRT1, Q923E4; SIRT2, Q8VDQ8; SIRT3, Q8R104; SIRT4, Q8R216; SIRT5, Q8K2C6; SIRT6, P59941; SIRT7, Q8BKJ9), rat (SIRT2, Q5RJQ4; SIRT5, Q68FX9), bovine (SIRT4, Q1JQC6; SIRT5, Q3ZBQ0; SIRT7, Q0P595), and others, as well as any sequence variants thereof due to normal sequence polymorphism between and within populations of each respective species.

The term encompasses sirtuins which comprise NAD⁺-dependent deacetylase activity (such as, e.g., SIRT1, 2, 3 and 5) and which can deacetylate histones and/or other protein substrates and typically concomitantly produce nicotinamide and 2′-O- or 3′-O-acetyl-ADP ribose (AADPR). The term also encompasses sirtuins which comprise NAD⁺-dependent ADP-ribosyl transferase activity (such as, e.g., SIRT4 and 6). The term may thus encompass sirtuins comprising any one, two or more of: (a) NAD⁺-dependent deacetylase activity, (b) AADPR-generating activity and (c) ADP-ribosyl transferase activity. However, the term may also encompass sirtuins for which none of the above enzymatic activities has (as yet) been demonstrated and/or which may comprise one or more further, as yet unidentified, enzymatic activities.

The term “sirtuin inhibitor” generally encompasses (1) agents capable of reducing the level of expression of one or more sirtuin members in cells, tissues, organs or organisms, and (2) agents capable of interfering with one or more biochemical or cell biological functions of one or more sirtuin proteins.

Exemplary but non-limiting agents under (1) above may comprise antisense oligonucleotides, constructs encoding antisense transcripts, or synthetic or recombinant RNAi agents, such as siRNA or shRNA or constructs encoding such, etc. Such nucleic acid reagents may usually comprise DNA, RNA, hybrid DNA/RNA or PNA, optionally including modified sugar moieties, e.g., 2′-O-methylated, 2′-O-ethylated or 2′-O, 4′-C-ethylated, and/or modified phosphate groups, e.g., phosphorothioate, phosphorodithioate, methylphosphonate, phosphoramidate, alkyl phosphotriester, sulfamate, 3′-thioacetal, methylene (methylimino), 3′-N-carbamate or morpholino carbamate. Preparation of such agents for genes having known sequences is established in the art. For instance, further details on RNAi agents are in Elbashir et al. 2001 (Nature 411: 494-501), Reynolds et al. 2004 (Nat Biotechnol 22: 326-30), http://maidesigner.invitrogen.com/rnaiexpress, Wang & Mu 2004 (Bioinformatics 20: 1818-20) and Yuan et al. 2004 (Nucleic Acids Res 32 (Web Server issue): W130-4).

Preferably, but without limitation, sirtuin inhibitors under (2) above may be able to inhibit one or more enzymatic activities of said one or more sirtuin proteins, particularly one or more enzymatic activities chosen from (a) NAD⁺-dependent deacetylase activity, (b) AADPR-generating activity or (c) ADP-ribosyl transferase activity. The extent of inhibition of these enzymatic activities can be quantitated using methods routinely known in the art, e.g., in vitro deacetylation assays (e.g., North et al. 2003. Mol Cell 11: 437-44), ADP-ribosylation assays (e.g., Liszt et al. 2005, supra), etc.

The term inhibitor encompasses both reversible inhibitors, i.e., ones binding non-covalently to an enzyme and/or to an enzyme-substrate complex, as well as irreversible inhibitors, i.e., ones that covalently alter one or more amino acids usually in the active site of an enzyme. Among reversible inhibitors, the term encompasses any types thereof including inter alia competitive inhibitors (I binds E but not ES, i.e., I competes with S for binding to E; ↑ Kₘ; ≈Vₘₐₓ), non-competitive inhibitors (I binds both E and ES with identical affinities Kᵢ=K_{i′}; EIS does not have catalytic activity; ≈Kₘ; ↓ Vₘₐₓ), mixed-type inhibitors (I binds both E and ES with different affinities Kᵢ≠K_{i′}; EIS does not have catalytic activity; ↑ Kₘ; ↓ Vₘₐₓ), partially competitive inhibitors (I binds both E and ES with identical affinities Kᵢ=K_{i′}; EIS retains catalytic activity albeit lower than ES; ≈Kₘ; ↓ Vₘₐₓ), and uncompetitive inhibitors (I binds ES but not E; EIS is catalytically inactive; ↓ Kₘ; ↓ Vₘₐₓ). The information in parentheses summarises the interactions of the different inhibitor types with and their effect on the enzyme or enzyme-substrate complex, as well the consequences of the presence of the respective inhibitors for the Michaelis-Menten kinetic constants (Vₘₐₓ, Kₘ) of the enzyme. “E” denotes enzyme; “I” denotes inhibitor; “S” denotes a substrate of the enzyme; “ES” denotes enzyme-substrate complex; “EIS” denotes enzyme-inhibitor-substrate complex; Kᵢ is the dissociation constant of the EI complex; K_{i′} is the dissociation constant of the EIS complex; Kₘ denotes the Michaelis constant, i.e., substrate concentration at which the rate of the enzyme reaction is ½ Vₘₐₓ; Vₘₐₓ denotes the enzyme's maximum reaction rate (the kinetic constants Vₘₐₓ and Kₘ have a well-established meaning in standard kinetic analysis); ≈means remains substantially same; ↓ means decreases; ↑ means increases. Hence, an increase in Kₘ generally suggests that an inhibitor interferes with binding of substrate to the enzyme, while a decrease in Vₘ generally suggests that binding of an inhibitor to the ES complex hampers catalysis.

Preferably, a reversible inhibitor (I) can strongly bind to at least one of enzyme (E) (e.g., a competitive inhibitor) or enzyme-substrate complex (ES) (e.g., an uncompetitive inhibitor), and possibly to both enzyme (E) and enzyme-substrate complex (ES) (e.g., a non-competitive inhibitor). As used herein, “strongly bind” means that the dissociation constant of the respective binding (Kᵢ or K_{i′}) is <1×10⁻⁴ M, preferably ≦1×10⁻⁵ M, even more preferably ≦1×10⁻⁶ M, such as, e.g., ≦1×10⁻⁷ M, yet more preferably ≦1×10⁻⁵ M, even more preferably ≦1×10⁻⁹ M, e.g., ≦1×10⁻¹⁰ M, and very preferably ≦1×10⁻¹¹ M, e.g., ≦1×10⁻¹² M, ≦1×10⁻¹³ M, ≦1×10⁻¹⁴ M, ≦1×10⁻¹⁵ M or even less, wherein Kᵢ=[E][I]/[EI] and K_{i′}=[ES][I]/[ESI]. Determination of Kᵢ may be carried out directly by, e.g., isothermal titration calorimetry (Holdgate 2001. Biotechniques 31: 164-6). Kᵢ and/or K_{i′} may be measured indirectly, by observing the enzyme activity under various substrate and inhibitor concentrations and fitting the data to a modified Michaelis-Menten equation, as known in the art.

Preferably, a sirtuin inhibitor according to the invention is specific, i.e., while inhibiting one or more biochemical or cell biological functions, e.g., one or more enzymatic activities, of the corresponding one or more sirtuin members, it does not substantially affect other biological components of cells, tissues, organs or organisms, and in particular other enzyme systems, such that effect(s) on downstream cellular components and functions are a direct consequence of the impact of the inhibitor on the one or more sirtuin members.

For example, sirtuin inhibitors may take the form of inter alia a chemical substance, a pharmaceutical agent or drug, a nucleic acid agent, a specific binding agent, a fragment or variant of sirtuins, e.g., dominant negative forms thereof, etc. Preferably, a sirtuin inhibitor may be a small organic molecule, more preferably having molecular mass up to about 5000 Da, e.g., up to about 4000, preferably up to about 3000 Da, more preferably up to 2000 Da, even more preferably up to about 1000 Da, e.g., up to about 900, 800, 700, 600 or up to about 500 Da.

By means of example and not limitation, sirtuin inhibitors in any of the herein disclosed aspects may comprise sirtinol (2-[(2-Hydroxynaphthalen-1-ylmethylene)amino]-N-(1-phenethyl)benzamide) (Grozinger et al. 2001. J Biol Chem 276: 38837-43); compounds A3 and M15 (Grozinger et al. 2001, supra); m- and p-sirtinol (respectively, 3- and 4-[(2-hydroxy-1-naphthalenylmethylene)amino]-N-(1-phenylethyl)benzamide) (Mai at al. 2005. J Med Chem 48: 7789); splitomycin (1,2-Dihydro-3H-naphtho[2,1-b]pyran-3-one) (Bedalov et al. 2001. PNAS 98: 15113); dehydrosplitomycin and compound 26 (Hirao et al. 2003. J Biol Chem 278: 52773); cambinol (5-((2-hydroxy-1-naphthyl)methyl)-2-mercapto-6-phenyl-4-pyrimidinol) (Heltweg et al. 2006. Cancer Res 66: 4368-77); dihydrocoumarin (Olaharski et al. 2005. PLoS Genet 1: e77); as well as any, preferably pharmaceutically acceptable, derivatives thereof, e.g., esters, amides, N-oxides, addition salts or quaternary amines thereof, insofar they possess sirtuin inhibitory activity, preferably substantially identical to said molecules.

The term “cell-permeable” refers to compounds, such as sirtuin inhibitors, that are able to cross or permeate the cell membrane of live cells. Optionally, compounds may be rendered cell-permeable or their cell-permeability may be enhanced by lipophilic groups, such as, e.g., acetoxymethyl (AM) ester or acetate esters.

As noted in the Summary section, an aspect of the invention provides a method for reducing the production of TNF-alpha in cells in vitro, comprising exposing said cells to an inhibitor of one or more sirtuins.

The term “in vitro” denotes outside, or external to, animal or human body. The term “in vitro” as used herein typically refers to tissues or cells removed from an animal or human body and maintained and/or propagated (i.e., cultured) outside the body, e.g., in a culture vessel.

The term “cells” may, without limitation, encompass primary cells, secondary cells, as well as transformed or immortalised cell lines. The term “primary cells” includes: cells present in a tissue, organ, or a part thereof, removed from an organism; cells present in a suspension of cells obtained by disassociation of said tissue, organ, or part thereof; cells present in an explanted tissue; cells of said suspension or said explant when first time cultured; and cells present in a suspension of cells derived from the first time cultured cells. The term “secondary cell” refers to cells at all subsequent steps in cultivation. Hence, when primary cells cultured for the first time are passaged, they are thereafter referred to herein as secondary cells, as are all cells in subsequent passages. Transformed or immortalised cell lines do not manifest considerable senescence in culture and can undergo cell divisions in excess of the Hayflick limit, e.g., more than about 60 cell divisions and usually more than 70, 80, or more than 90 cell divisions, or many more. Such transformation may be achieved either spontaneously in culture or may be achieved by using transforming proteins, e.g., the large T antigen of SV40 virus, as established in the art. The so cultured cells may be optionally further genetically modified to express one or more biological substances of interest, e.g., one or more nucleic acids or proteins (e.g., preferably with TNF-alpha), as known in the art.

In an embodiment, said cells may preferably originate from or may descend from cells that have originated from an animal, more preferably from a warm-blood animal, even more preferably from a mammal, such as, very preferably, from human or from non-human mammal.

The cells may encompass any cell type. In an embodiment, the cells may encompass any cell type transiently or stably transformed with a recombinant nucleic acid construct capable of inducibly or constitutively expressing TNF-alpha in said cell type. In a further embodiment, the cells may encompass any cell type capable of constitutively or inducibly (e.g., in response to exposure to one or more cytokines) express endogenous TNF-alpha. In a preferred embodiment, the cells may encompass any cell type which normally participates, or are derived from a cell type which normally participates, in innate or adaptive immunity, preferably wherein said cell type is chosen from monocytes, macrophages, dendritic cells (DC), neutrophils, mast cells, eosinophils, basophils, eosinophils, natural killer (NK) cells, lymphokine activated killer (LAK) cells, T lymphocytes (including, e.g., T helper cells, cytotoxic T cells and γδ T cells) or B cells; more preferably chosen from monocytes, macrophages, dendritic cells (DC), neutrophils, mast cells, NK cells, LAK cells or T lymphocytes; even more preferably chosen from monocytes, macrophages, dendritic cells (DC), and neutrophils; such as, very preferably, chosen from macrophages or dendritic cells (DC).

The term “expose” or “exposing” means bringing cells in contact with the agent to which they are said to be exposed. Preferably, said exposure may be achieved by including said agent in one or more reagents used for isolating the cells and/or to one or more culture media used to culture said cells.

Exposure of said cells to an inhibitor of one or more sirtuins may cause any extent of reduction in the expression or biochemical or cell biological activity of said one or more sirtuin members. Preferably, said expression or biochemical or cell biological activity of said one or more sirtuin members may be reduced by at least about 10%, e.g., by at least about 20%, more preferably by at least about 30%, e.g., by at least about 40%, even more preferably by at least about 50%, e.g., by at least 60%, still more preferably by at least 70%, e.g., by at least about 80%, and very preferably by at least about 90%, e.g., by at least about 95% or by about 100%, relative to the basal level, i.e., the level of expression or biochemical or cell biological activity of said one or more sirtuin members in cells without (e.g., prior to) exposure to said inhibitor.

As noted in the Summary section, further aspects of the invention relate to application of sirtuin inhibitors in modulating conditions or states in subjects, e.g., for reducing—locally or systemically—the production of TNF-alpha in subjects, for reducing local or systemic inflammation in a subject, or for treatment of TNF-alpha mediated pathologies.

“Subject” or “patient” as used herein refer to animals, preferably vertebrates, more preferably warm-blooded vertebrates, even more preferably mammals, and very preferably human patients and non-human mammal subjects. The term “mammal” includes any animal classified as such, including, but not limited to, humans, domestic and farm animals, zoo animals, sport animals, pet animals, companion animals and experimental animals, such as, for example, mice, rats, hamsters, rabbits, dogs, cats, guinea pigs, cattle, cows, sheep, horses, pigs and primates, e.g., monkeys and apes.

Preferred patients are human subjects.

As used herein, the terms “treat” or “treatment” refer to both therapeutic treatment and prophylactic or preventative measures, wherein the object is to prevent or slow down (lessen) an undesired physiological change or disorder, such as the development or progression of a TNF-alpha mediated, disorder. Beneficial or desired clinical results include, but are not limited to, alleviation of one or more symptoms or one or more biological markers (e.g., symptoms of inflammation, e.g., heat, fever, swelling, pain, loss of function, etc.), diminishment of extent of disease, stabilised (i.e., not worsening) state of disease, delay or slowing of disease progression, amelioration or palliation of the disease state, prolongation of time between relapses, etc. “Treatment” can also mean prolonging survival as compared to expected survival if not receiving treatment.

As used herein, a phrase such as “a subject in need of treatment” includes subjects, such as mammalian subjects, more preferably human subjects, that would benefit from treatment of a given condition, more particularly from reducing locally or systemically the production of TNF-alpha, from reducing local or systemic inflammation, and/or from treatment of TNF-alpha mediated pathologies. Such subjects will typically include, without limitation, those that have been diagnosed with the condition, those prone to have or develop the said condition and/or those in whom the condition is to be prevented.

The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of the disease being treated.

The term “inflammation” as used herein has the denotation given to it in the art. By means of further guidance, the term generally refers to a (typically local) response in vasculated tissues to cellular or tissue injury usually caused by physical, chemical and/or biological agents, that is marked in the acute form by the classical sequences of pain, heat, redness, swelling, and loss of function, and serves as a mechanism initiating the elimination, dilution or walling-off of noxious agents and/or of damaged tissue. Inflammation histologically involves a complex series of events, including dilation of the arterioles, capillaries, and venules with increased permeability and blood flow, exudation of fluids including plasma proteins, and leukocyte migration into the inflammatory focus.

Further, the term encompasses inflammation caused by extraneous physical or chemical injury or by biological agents, e.g., viruses, bacteria, fungi, protozoan or metazoan parasite infections, as well as inflammation which is seemingly unprovoked, e.g., which occurs in the absence of demonstrable injury or infection, inflammation responses to self-antigens (auto-immune inflammation), inflammation responses to engrafted xenogenic or allogeneic cells, tissues or organs, inflammation responses to allergens, etc. The term covers both acute inflammation and chronic inflammation. Also, the term includes both local or localised inflammation, as well as systemic inflammation, i.e., where one or more inflammatory processes are not confined to a particular tissue but occur generally in the endothelium and/or other organ systems.

As noted, TNF-alpha mediated pathologies may be at least in part contributed to by local or systemic excessive or deregulated production of TNF-alpha in subjects, which may, for example, exacerbate various acute or chronic inflammatory states in the subjects.

In an embodiment, a TNF-alpha mediated pathology may be characterised by a statistically significantly increased steady-state level and/or rate of production of soluble and/or membrane bound TNF-alpha, preferably at least or only soluble TNF-alpha, by cells, tissues, organs or system of a subject. Any extent of such increase is contemplated, and may preferably be by at least about 10%, e.g., by at least about 20%, by at least about 30%, by at least about 40%, more preferably by at least about 50%, e.g., by at least about 60%, by at least about 70%, by at least about 80% by at least about 90%, even more preferably by at least about 100%, e.g., by at least about 150%, or even by at least about 200%, at least about 400% or more, relative to the basal TNF-alpha level or production rate by said cells, tissues, organs or system in physiologically normal subjects.

The following preferred embodiments list disorder, the prevention or treatment of which is contemplated using sirtuin inhibitor(s) according to the invention, such as various TNF-alpha mediated pathologies.

In an embodiment, the invention contemplates the prevention or treatment of cachexia, e.g., cachexia associated with cancer or infectious diseases, such as, e.g., AIDS.

In another embodiment, the invention contemplates the prevention or treatment of a condition chosen from: Gram-negative sepsis, endotoxin-induced shock, septic shock syndrome, systemic inflammatory response syndrome (SIRS) or multiple organ dysfunction syndrome (MODS).

In a further embodiment, the invention contemplates the prevention or treatment of graft versus host pathologies, such as, e.g., graft versus host disease (GVHD) or rejection of transplanted xenogenic or allogeneic tissues or organs, such as, e.g., rejection of allogeneic bone marrow or cord blood transplants.

In yet other embodiments, the invention contemplates the prevention or treatment of acute and chronic infectious and parasitic processes, such as viral, bacterial or fungal, infections and protozoan or metazoan parasites, including, preferably, cerebral malaria or meningococcal meningitis.

In a further preferred embodiment, the invention contemplates the prevention or treatment of allergic disorders, such as, e.g., allergic rhinitis, allergic conjunctivitis, asthma, eczema, urticaria, contact dermatitis, systemic allergic response (anaphylaxis) or anaphylactic shock, more preferably chosen from allergic rhinitis and asthma.

In a particularly preferred embodiment, the invention contemplates the prevention or treatment of chronic inflammatory disorders (generally encompassing a heterogeneous group of conditions typically involving chronic or recurrent local or systemic activity of one or more inflammatory processes and/or components of innate or adaptive immunity in the absence of demonstrable cause, e.g., infection or tissue injury) and/or autoimmune diseases (generally involving immune response against a self tissue or tissue component, i.e., self-antigen, including a self antibody response or cell-mediated response, and also including organ-specific and non-organ specific autoimmune conditions.

Preferably, such condition may be chosen from: acute disseminated encephalomyelitis (ADEM); Addison's disease; ankylosing spondylitis; antiphospholipid antibody syndrome (APS); aplastic anemia; atherosclerosis; autoimmune gastritis; autoimmune hepatitis; autoimmune thrombocytopenia; Behcet's disease; coeliac disease; dermatomyositis; diabetes mellitus type I; diabetes mellitus type II; familial Mediterranean fever; familial cold-induced autoinflammatory syndrome; Goodpasture's syndrome; gout; pseudogout; Graves' disease; Guillain-Barré syndrome (GBS); Hashimoto's disease; hereditary periodic fevers; idiopathic thrombocytopenic purpura; inflammatory bowel disease (IBD) including Crohn's disease and ulcerative colitis; ischemia-reperfusion injury; Kawasaki's disease; mixed connective tissue disease; Muckle-Wells syndrome; multiple sclerosis (MS); myasthenia gravis; opsoclonus myoclonus syndrome (OMS); optic neuritis; Ord's thyroiditis; osteoarthritis; pemphigus; pernicious anaemia; polyarteritis nodosa; polymyositis; postoperative or traumatic inflammation; primary biliary cirrhosis; primary myoxedema; psoriasis; psoriatic arthritis; rheumatic fever; rheumatoid arthritis; Reiter's syndrome; scleroderma; Sjögren's syndrome; stroke-ischemia; systemic lupus erythematosus (SLE); systemic onset juvenile idiopathic arthritis; Takayasu's arteritis; temporal arteritis; vitiligo; warm autoimmune hemolytic anemia; and Wegener's granulomatosis.

In a preferred embodiment, said condition may be chosen from: ankylosing spondylitis; atherosclerosis; diabetes mellitus type II; inflammatory bowel disease (IBD) including Crohn's disease and ulcerative colitis; ischemia-reperfusion injury; multiple sclerosis (MS); psoriasis; psoriatic arthritis; rheumatic fever; rheumatoid arthritis; stroke-ischemia; systemic lupus erythematosus (SLE).

In a further preferred embodiment, said condition may be chosen from: ankylosing spondylitis; atherosclerosis; inflammatory bowel disease (IBD) including Crohn's disease and ulcerative colitis; psoriasis; psoriatic arthritis; and rheumatoid arthritis.

As described herein, the beneficial effects on TNF-alpha production, inflammation and/or TNF-alpha mediated conditions is achieved in cells or subjects using sirtuin inhibitors, preferably specific sirtuin inhibitors.

In an embodiment, administration of an inhibitor of one or more sirtuins to a subject may cause any extent of reduction, locally or systemically, in the expression or biochemical or cell biological activity of said one or more sirtuin members. Preferably, said expression or biochemical or cell biological activity of said one or more sirtuin members may be reduced by at least about 10%, e.g., by at least about 20%, more preferably by at least about 30%, e.g., by at least about 40%, even more preferably by at least about 50%, e.g., by at least 60%, still more preferably by at least 70%, e.g., by at least about 80%, and very preferably by at least about 90%, e.g., by at least about 95% or by about 100%, relative to the basal level, i.e., the level of expression or biochemical or cell biological activity of said one or more sirtuin members in cells without (e.g., prior to) administration of said inhibitor.

In a preferred embodiment, the inhibitor may specifically inhibit one or more sirtuin chosen from the group comprising or consisting of SIRT1, SIRT2, SIRT3, SIRT4, SIRT5, SIRT6 and SIRT7.

In a particularly preferred embodiment, the inhibitor may inhibit, preferably specifically, SIRT1 and/or SIRT6. The inventors have realised that SIRT1 and SIRT6 sirtuins have a particularly pronounced effect on the production of TNF-alpha, and thus may represent, alone or in combination, preferred targets for inhibition in TNF-alpha mediated conditions.

Particularly preferably the sirtuin inhibitor may inhibit SIRT6; at least SIRT6; or substantially only (i.e., selectively) SIRT6. Selective inhibition of SIRT6 may for example encompass at least 10-fold greater, or at least 100-fold greater or at least 1000-fold greater inhibition of SIRT6 than of other SIRT. The inventors have realised that SIRT6 has a particularly pronounced effect on the production of TNF-alpha, and thus may represent preferred target for inhibition in TNF-alpha mediated conditions.

In further embodiment, the inhibitor may interfere with one or more biochemical or cell biological functions of the sirtuin protein. The recitation biochemical or cell biological functions is contemplated broadly herein and refers to any and all functions which the respective sirtuin protein may have in cells or tissues.

In a preferred embodiment, the inhibitor may inhibit with one or more (such as, e.g., one) enzymatic activities of the sirtuin protein, and preferably wherein said one or more enzymatic activities are chosen from (a) NAD⁺-dependent deacetylase activity, (b) AADPR-generating activity and (c) ADP-ribosyl transferase activity. In a preferred embodiment, the inhibitor may inhibit at least the NAD⁺-dependent deacetylase activity and/or the AADPR-generating activity of the sirtuin protein. In another preferred embodiment, the inhibitor may inhibit at least the ADP-ribosyl transferase activity of the sirtuin protein.

In an embodiment, the inhibitor may be a reversible inhibitor or an irreversible inhibitor, more preferably a reversible inhibitor. Preferably, said inhibitor may achieve competitive, non-competitive or mixed-type inhibition of the sirtuin protein.

In a preferred embodiment, sirtuin inhibitor for use in the invention may be chosen from the group comprising or consisting of sirtinol; compound A3 and compound and M15 as disclosed by Grozinger et al. 2001, supra; m-sirtinol; p-sirtinol; splitomycin; dehydrosplitomycin; compound 26 as disclosed by Hirao et al. 2003, supra; cambinol; and dihydrocoumarin. More preferably, sirtuin inhibitor for use in the invention may be chosen from the group comprising or consisting of sirtinol; m-sirtinol; p-sirtinol; splitomycin; dehydrosplitomycin; cambinol; and dihydrocoumarin. Even more preferably, sirtuin inhibitor for use in the invention may be chosen from the group comprising or consisting of sirtinol; splitomycin; cambinol and dihydrocoumarin.

Sirtuin inhibitors, including but not limited to those recited in the previous paragraph, may be employed, e.g., administered to subjects, as such or in the form of pharmaceutically acceptable derivatives, e.g., esters, amides, N-oxides, addition salts or quaternary amines, insofar these possess sirtuin inhibitory activity, preferably substantially identical to the inhibitors as such.

The term “pharmaceutically acceptable” as used herein is consistent with the art and means compatible with the other ingredients of a pharmaceutical composition and not deleterious to the recipient thereof.

The term “addition salts”, in particular “pharmaceutically acceptable addition salts” is meant to include salts of sirtuin inhibitors which are prepared with relatively non-toxic acids or bases, depending on the particular substituents found on the inhibitors.

When sirtuin inhibitors contain relatively acidic functionalities, base addition salts can be obtained by contacting the neutral form of such inhibitors with a sufficient amount of the desired base, either neat or in a suitable inert solvent. Examples of pharmaceutically acceptable base addition salts include alkali metal salts, such as, e.g., lithium, sodium or potassium salts, alkaline earth metal salts, such as, e.g., calcium or magnesium salts, and further aluminum salts, zinc salts, ammonium salts and organic amino salts, such as, e.g., tetramethylammonium salts, tetraethylammonium salts, salts with morpholine or piperidine, or salts with amino acids, such as, e.g., salts with lysine, arginine, glycine or phenylalanine, or the like bases. Conversely, said base addition salt forms can be converted by treatment with an appropriate acid into the free acid form of the inhibitors.

When sirtuin inhibitors contain relatively basic functionalities, acid addition salts can be obtained by contacting the neutral form of such inhibitors with a sufficient amount of the desired acid, either neat or in a suitable inert solvent. Examples of pharmaceutically acceptable acid addition salts include those derived from inorganic acids, such as, e.g., hydrochloric, hydrobromic, nitric, carbonic, monohydrogencarbonic, phosphoric, monohydrogenphosphoric, dihydrogenphosphoric, sulphuric, monohydrogensulphuric, hydriodic or phosphorous acids or the like, as well as salts derived from relatively non-toxic organic acids, such as, e.g., acetic, hydroxyacetic, propionic, isobutyric, maleic, malonic, benzoic, succinic, suberic, fumaric, lactic, pyruvic, oxalic, malic, mandelic, phthalic, benzenesulfonic, p-tolylsulphonic, citric, tartaric, methanesulphonic, ethanesulphonic, salicylic, p-amino-salicylic, glucuronic or galactunoric acids or the like acids. See, e.g., Berge et al. (“Pharmaceutical Salts”, J Pharm Sci 66: 1-19). Conversely said acid addition salt forms can be converted by treatment with an appropriate base into the free base form of the inhibitors. Some sirtuin inhibitors may contain both basic and acidic functionalities that allow the inhibitors to be converted into either base or acid addition salts.

In the present invention, the sirtuin inhibitors or pharmaceutically acceptable derivatives thereof may be administered as such or, preferably, in the form of pharmaceutical compositions obtained by formulating the inhibitors or derivatives thereof with per se known pharmaceutically acceptable carriers/excipients.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antibacterial and antifungal agents, preservatives, antioxidants, tonicity controlling agents, absorption delaying agents, and the like. The use of such media and agents for pharmaceutical active substances is well known in the art. Except insofar as any conventional media or agent is incompatible with the active substance, its use in the therapeutic compositions may be contemplated.

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, microspheres, microbeads and microsomes, powders, tablets, capsules, suppositories, aqueous suspensions, aerosols, and other carriers apparent to one of ordinary skill in the art.

Pharmaceutical compositions of the invention may be formulated for essentially any route of administration, such as without limitation, oral administration (such as, e.g., oral ingestion or inhalation), intranasal administration (such as, e.g., intranasal inhalation or intranasal mucosal application), parenteral administration (such as, e.g., subcutaneous, intravenous, intramuscular, intraperitoneal or intrasternal injection or infusion), transdermal or transmucosal (such as, e.g., oral, sublingual, intranasal) administration, rectal, vaginal or intra-tracheal instillation, and the like. In this way, the therapeutic effects attainable by the methods and compositions of the invention can be, for example, systemic, local, tissue-specific, etc., depending of the specific needs of a given application of the invention.

For example, for oral administration, pharmaceutical compositions may be formulated in the form of pills, tablets, lacquered tablets, coated (e.g., sugar-coated) tablets, granules, hard and soft gelatin capsules, aqueous, alcoholic or oily solutions, syrups, emulsions or suspensions. In an example, without limitation, preparation of oral dosage forms may be is suitably accomplished by uniformly and intimately blending together a suitable amount of the active compound in the form of a powder, optionally also including finely divided one or more solid carrier, and formulating the blend in a pill, tablet or a capsule. Exemplary but non-limiting solid carriers include calcium phosphate, magnesium stearate, talc, sugars (such as, e.g., glucose, mannose, lactose or sucrose), sugar alcohols (such as, e.g., mannitol), dextrin, starch, gelatin, cellulose, polyvinylpyrrolidine, low melting waxes and ion exchange resins. Compressed tablets containing the pharmaceutical composition can be prepared by uniformly and intimately mixing the active ingredient with a solid carrier such as described above to provide a mixture having the necessary compression properties, and then compacting the mixture in a suitable machine to the shape and size desired. Moulded tablets maybe made by moulding in a suitable machine, a mixture of powdered compound moistened with an inert liquid diluent. Suitable carriers for soft gelatin capsules and suppositories are, for example, fats, waxes, semisolid and liquid polyols, natural or hardened oils, etc.

For example, for oral or nasal aerosol or inhalation administration, pharmaceutical compositions may be formulated with illustrative carriers, such as, e.g., as in solution with saline, polyethylene glycol or glycols, DPPC, methylcellulose, or in mixture with powdered dispersing agents, further employing benzyl alcohol or other suitable preservatives, absorption promoters to enhance bioavailability, fluorocarbons, and/or other solubilising or dispersing agents known in the art. Suitable pharmaceutical formulations for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Illustratively, delivery may be by use of a single-use delivery device, a mist nebuliser, a breath-activated powder inhaler, an aerosol metered-dose inhaler (MDI) or any other of the numerous nebuliser delivery devices available in the art. Additionally, mist tents or direct administration through endotracheal tubes may also be used.

Examples of carriers for administration via mucosal surfaces depend upon the particular route, e.g., oral, sublingual, intranasal, etc. When administered orally, illustrative examples include pharmaceutical grades of mannitol, starch, lactose, magnesium stearate, sodium saccharide, cellulose, magnesium carbonate and the like, with mannitol being preferred. When administered intranasally, illustrative examples include polyethylene glycol, phospholipids, glycols and glycolipids, sucrose, and/or methylcellulose, powder suspensions with or without bulking agents such as lactose and preservatives such as benzalkonium chloride, EDTA. In a particularly illustrative embodiment, the phospholipid 1,2 dipalmitoyl-sn-glycero-3-phosphocholine (DPPC) is used as an isotonic aqueous carrier at about 0.01-0.2% for intranasal administration of the sirtuin inhibitor of the invention at a concentration of about 0.1 to 3.0 mg/ml.

For example, for parenteral administration, pharmaceutical compositions may be advantageously formulated as solutions, suspensions or emulsions with suitable solvents, diluents, solubilisers or emulsifiers, etc. Suitable solvents are, without limitation, water, physiological saline solution or alcohols, e.g. ethanol, propanol, glycerol, in addition also sugar solutions such as glucose, invert sugar, sucrose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. The compounds and pharmaceutically acceptable salts thereof of the invention can also be lyophilised and the lyophilisates obtained used, for example, for the production of injection or infusion preparations. For example, one illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

Where aqueous formulations are preferred, such may comprise one or more surfactants. For example, the composition can be in the form of a micellar dispersion comprising at least one suitable surfactant, e.g., a phospholipid surfactant. Illustrative examples of phospholipids include diacyl phosphatidyl glycerols, such as dimyristoyl phosphatidyl glycerol (DPMG), dipalmitoyl phosphatidyl glycerol (DPPG), and distearoyl phosphatidyl glycerol (DSPG), diacyl phosphatidyl cholines, such as dimyristoyl phosphatidylcholine (DPMC), dipalmitoyl phosphatidylcholine (DPPC), and distearoyl phosphatidylcholine (DSPC); diacyl phosphatidic acids, such as dimyristoyl phosphatidic acid (DPMA), dipahnitoyl phosphatidic acid (DPPA), and distearoyl phosphatidic acid (DSPA); and diacyl phosphatidyl ethanolamines such as dimyristoyl phosphatidyl ethanolamine (DPME), dipalmitoyl phosphatidyl ethanolamine (DPPE) and distearoyl phosphatidyl ethanolamine (DSPE). Typically, a surfactant:active substance molar ratio in an aqueous formulation will be from about 10:1 to about 1:10, more typically from about 5:1 to about 1:5, however any effective amount of surfactant may be used in an aqueous formulation to best suit the specific objectives of interest.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquidify and/or dissolve in the rectal cavity to release the drug.

Suitable carriers for microcapsules, implants or rods are, for example, copolymers of glycolic acid and lactic acid.

One skilled in this art will recognize that the above description is illustrative rather than exhaustive. Indeed, many additional formulations techniques and pharmaceutically-acceptable excipients and carrier solutions are well-known to those skilled in the art, as is the development of suitable dosing and treatment regimens for using the particular compositions described herein in a variety of treatment regimens.

Sirtuin inhibitors may be used according to the invention alone or in combination with any art-known therapies to reduce TNF-alpha production or activity (e.g., monoclonal anti-TNF-alpha antibodies) and/or to generally diminish inflammation, such as, e.g., with anti-inflammatory agents, e.g., interferon beta-1a or beta-1b. Said additional agents disease agents can be administered before, after or simultaneously with the administration of the sirtuin inhibitors.

The dosage or amount of sirtuin inhibitors or derivatives thereof used, optionally in combination with one or more other active agents to be administered, depends on the individual case and is, as is customary, to be adapted to the individual circumstances to achieve an optimum effect. Thus, it depends on the nature and the severity of the disorder to be treated, and also on the sex, age, body weight, general health, diet, mode and time of administration, and individual responsiveness of the human or animal to be treated, on the route of administration, efficacy, metabolic stability and duration of action of the compounds used, on whether the therapy is acute or chronic or prophylactic, or on whether other active compounds are administered in addition to the agent(s) of the invention.

Without limitation, depending on the type and severity of the disease, a typical daily dosage might range from about 1 μg/kg to 100 mg/kg of body weight or more, depending on the factors mentioned above. For repeated administrations over several days or longer, depending on the condition, the treatment is sustained until a desired suppression of disease symptoms occurs. A preferred daily dosage of the sirtuin inhibitor or derivative thereof may be in the range from about 0.05 mg/kg to about 10 mg/kg of body weight. Thus, one or more doses of about 0.5 mg/kg, 2.0 mg/kg, 4.0 mg/kg or 10 mg/kg (or any combination thereof) may be administered to the patient. Such doses may also be administered intermittently, e.g., every several days, e.g., every two, three, four, five or six days, or every week, every two weeks or every three weeks, and the like.

In a further aspect, the invention also provides a pharmaceutical kit comprising: (1) a sirtuin inhibitor or pharmaceutically acceptable derivative thereof or a pharmaceutical composition comprising a sirtuin inhibitor or pharmaceutically acceptable derivative thereof and one or more pharmaceutically acceptable carrier, and (2) written, e.g., printed instructions to use of (1) in the treatment of TNF-alpha mediated conditions as taught herein.

The above aspects and embodiments are further supported by the following examples which are in no instance to be considered limiting.

### EXAMPLES

### Example 1

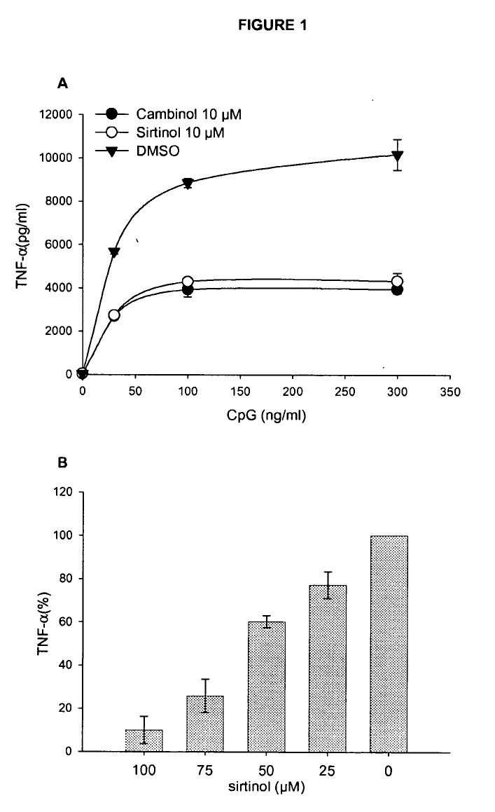

Sirtuin Inhibitors Dowregulate TNF-α Secretion (FIG. 1)

Murine dendritic cells (FIG. 1A) or the murine macrophage-like cell line RAW264.7 cells (FIG. 1B) were cultured in the presence of sirtuin inhibitors as indicated or an equivalent amount of solvent (DMSO) and stimulated overnight with graded doses of CpG (FIG. 1A) or for 2 h with 100 ng/ml of lipopolysaccharide (LPS) from gram-negative bacteria. Cell supernatants were tested for tumor necrosis factor (TNF-alpha) content by ELISA. The figure demonstrates that sirtuin inhibitors donwregulate TNF-alpha secretion induced by microbial compounds.

### Example 2

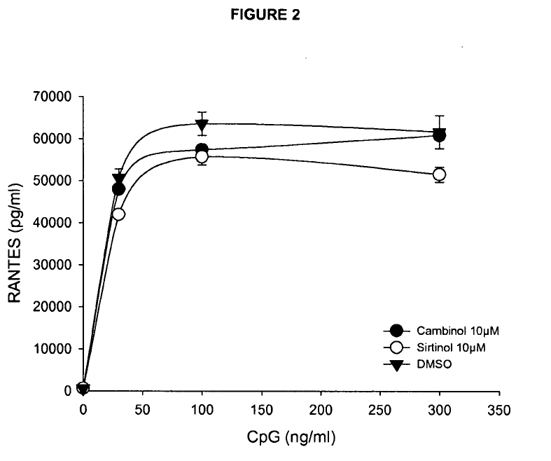

Downregulation of TNF-α Cytokine Secretion by Sirtuin Inhibitors is not Due to General Inhibition of Protein Synthesis or Induction of Cell Death (FIG. 2)

Sirtuin inhibitors failed to inhibit the secretion of RANTES, a pro-inflammatory chemokine secreted upon stimulation by microbial compounds. Murine dendritic cells were cultured in the presence of sirtuin inhibitors as indicated and stimulated overnight with graded doses of CpG. Cell supernatants were tested for RANTES content by ELISA. This experiment demonstrates that sirtuin inhibitors do not inhibit RANTES secretion in response to CpG, indicating that these compounds do not inhibit the protein synthesis machinery of the cell in a non-specific fashion, nor affect cell viability in culture.

### Experiment 3

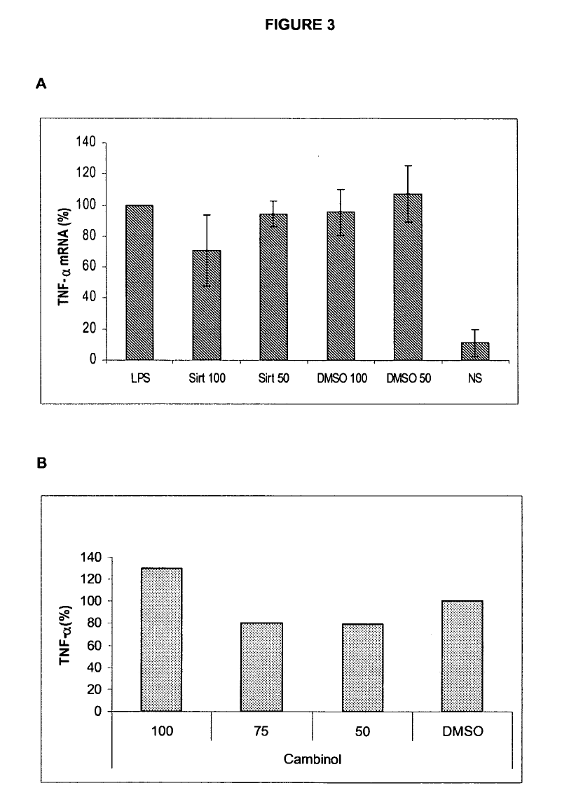

Sirtuin Inhibitors Downregulate TNF-α Cytokine Production without Affecting the Intracellular Accumulation of TNF-Alpha mRNA (FIG. 3).

RAW264.7 were incubated in the presence of sirtuin inhibitors or equivalent doses of solvent (DMSO) as indicated and stimulated for 2 h by LPS. Expression of mRNA encoding for TNF-α was monitored using quantititative RT-PCR using standard procedures and the following forward and reverse primers (fwd: gcctccctctcatcagttcta; rev: gctacgacgtgggctacag). The present experiment demonstrates that while sirtuin inhibitors (sirtinol, cambinol) inhibit TNF-α protein synthesis (see FIG. 1 as an example), they do not significantly affect TNF-alpha mRNA accumulation in response to microbial compounds. Collectively, these observations indicate that sirtuin inhibitors may affect TNF-alpha secretion by interfering with at a post-transcriptional step.

### Experiment 4

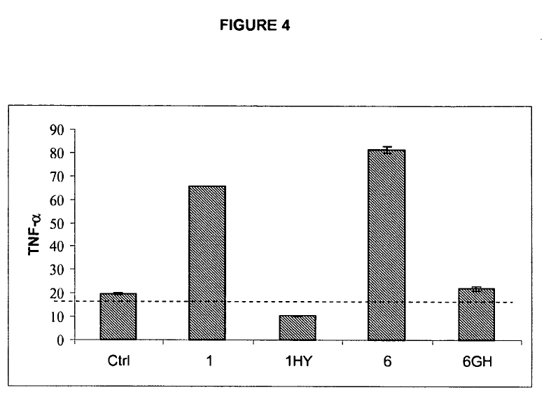

Overexpression of SIRT1 and SIRT6 Increases Constitutive TNF-α Cytokine Production by Cell Lines Transiently Transfected with a TNF-α-Encoding Plasmid (FIG. 4)

Human Hela cells were co-transfected with plasmid encoding for selected sirtuin members and with a plasmid encoding for TNF-alpha. Plasmid used in this experiment encoded for wild type murine SIRT1 and SIRT6 or for catalytically inactive mutants (SIRT1 mutant designated 1HY and SIRT6 mutant designated 6GH) obtained following directed in vitro mutagenesis. Choice of mutants was based on published evidence (Khan A N, J Biol Chem. 2006 28; 281:11702). This figure demonstrates that overexpression of catalytically active, but not inactive mutants, SIRT1 and SIRT6 led to increased secretion of TNF-α when compared to control cells (designated as Ctrl, representing cells transiently transfected with a TNF-α-encoding plasmid). This experiment demonstrates that sirtuin members may promote TNF-α secretion in a catalytically-dependent fashion.

1. A method for reducing the production of TNF-alpha in cells in vitro, comprising exposing said cells to an inhibitor of one or more sirtuins other than suramin.

2. A method for reducing the production of TNF-alpha in cells in vitro, comprising exposing said cells to a cell-permeable inhibitor of one or more sirtuins.

3. The method according to any of claim 1 or 2, wherein the sirtuin inhibitor inhibits SIRT6.

4. The method according to any of claims 1 to 3 wherein the sirtuin inhibitor is chosen from sirtinol, m-sirtinol, p-sirtinol, splitomycin, dehydrosplitomycin, cambinol, and dihydrocoumarin.

5. The method according to any of claims 1 to 4, wherein said cells are able to constitutively or inducibly express endogenous TNF-alpha.

6. The method according to any of claims 1 to 5, wherein the cells are of a cell type, or are derived from a cell type, which normally participates in innate or adaptive immunity, preferably wherein said cell type is chosen from monocytes, macrophages, dendritic cells (DC), neutrophils, mast cells, eosinophils, basophils, eosinophils, natural killer (NK) cells, lymphokine activated killer (LAK) cells, T lymphocytes or B cells.

7. An inhibitor of one or more sirtuins other than suramin for (a) reducing the production of TNF-alpha in a subject and/or (b) reducing local or systemic inflammation in a subject and/or (c) treating a TNF-alpha mediated pathology in a subject having local or systemic excessive or deregulated production of TNF-alpha.

8. A cell-permeable inhibitor of one or more sirtuins for (a) reducing the production of TNF-alpha in a subject and/or (b) reducing local or systemic inflammation in a subject and/or (c) treating a TNF-alpha mediated pathology in a subject having local or systemic excessive or deregulated production of TNF-alpha.

9. An inhibitor of one or more sirtuins for use according to any of claim 7 or 8, wherein the sirtuin inhibitor inhibits SIRT6.

10. An inhibitor of one or more sirtuins for use according to any of claims 7 to 9, wherein the sirtuin inhibitor is chosen from sirtinol, m-sirtinol, p-sirtinol, splitomycin, dehydrosplitomycin, cambinol, and dihydrocoumarin.

11. An inhibitor of one or more sirtuins as defined in any of claims 7 to 10 for the treatment of:
cachexia; and/or
Gram-negative sepsis, endotoxin-induced shock, septic shock syndrome, systemic inflammatory response syndrome (SIRS) or multiple organ dysfunction syndrome (MODS); and/or
graft versus host pathologies, including graft versus host disease (GVHD) and rejection of transplanted xenogenic or allogeneic tissues or organs; and/or
acute or chronic infectious or parasitic processes, including viral, bacterial or fungal, infections and protozoan or metazoan parasite infections, preferably cerebral malaria or meningococcal meningitis; and/or
allergic disorders, including allergic rhinitis, allergic conjunctivitis, asthma, eczema, urticaria, contact dermatitis, systemic allergic response (anaphylaxis) and anaphylactic shock, preferably allergic rhinitis or asthma.

12. An inhibitor of one or more sirtuins as defined in any of claims 7 to 10 for the treatment of chronic inflammatory disorders and/or autoimmune diseases, preferably chosen from: acute disseminated encephalomyelitis (ADEM); Addison's disease; ankylosing spondylitis; antiphospholipid antibody syndrome (APS); aplastic anemia; atherosclerosis; autoimmune gastritis; autoimmune hepatitis; autoimmune thrombocytopenia; Behcet's disease; coeliac disease; dermatomyositis; diabetes mellitus type I; diabetes mellitus type II; familial Mediterranean fever; familial cold-induced autoinflammatory syndrome; Goodpasture's syndrome; gout; pseudogout; Graves' disease; Guillain-Barré syndrome (GBS); Hashimoto's disease; hereditary periodic fevers; idiopathic thrombocytopenic purpura; inflammatory bowel disease (IBD) including Crohn's disease and ulcerative colitis; ischemia-reperfusion injury; Kawasaki's disease; mixed connective tissue disease; Muckle-Wells syndrome; multiple sclerosis (MS); myasthenia gravis; opsoclonus myoclonus syndrome (OMS); optic neuritis; Ord's thyroiditis; osteoarthritis; pemphigus; pernicious anaemia; polyarteritis nodosa; polymyositis; postoperative or traumatic inflammation; primary biliary cirrhosis; primary myoxedema; psoriasis; psoriatic arthritis; rheumatic fever; rheumatoid arthritis; Reiter's syndrome; scleroderma; Sjögren's syndrome; stroke-ischemia; systemic lupus erythematosus (SLE); systemic onset juvenile idiopathic arthritis; Takayasu's arteritis; temporal arteritis; vitiligo; warm autoimmune hemolytic anemia; and Wegener's granulomatosis.

13. An inhibitor of one or more sirtuins as defined in any of claims 7 to 10 for the treatment of ankylosing spondylitis; atherosclerosis; inflammatory bowel disease (IBD) including Crohn's disease and ulcerative colitis; psoriasis; psoriatic arthritis; or rheumatoid arthritis.
